(19) United States
(12) Patent Application Publication
(10) Pub. No.: US 2021/0330907 A1
(43) Pub. Date: Oct. 28, 2021

(54) GAS EVACUATING PATIENT INTERFACE

(71) Applicant: KONINKLIJKE PHILIPS N.V., EINDHOVEN (NL)

(72) Inventors: Richard Thomas HAIBACH, VERONA, NY (US); Richard Andrew SOFRANKO, FINLEYVILLE, PA (US); Derrick Blake ANDREWS, MARKLETON, PA (US); Bernard F. HETE, KITTANNING, PA (US)

(21) Appl. No.: 17/204,115

(22) Filed: Mar. 17, 2021

Related U.S. Application Data
(60) Provisional application No. 63/015,944, filed on Apr. 27, 2020.

(51) Int. Cl. A61M 16/00; A61M 16/06; A61M 16/08

(52) U.S. Cl. CPC A61M 16/009; A61M 16/0622; A61M 16/0003; A61M 16/0683; A61M 16/0816

(57) ABSTRACT

A patient interface for use in delivering a flow of a breathing gas to an airway of a patient includes an inner mask having an inner cushion with an inward curving inner sealing portion that is structured to sealingly engage the face of the patient, and an outer mask coupled to the inner mask. The outer mask has an outer cushion with an outward curving outer sealing portion that is structured to sealingly engage the face of the patient completely around, and outward from, the inner sealing portion of the inner mask. The inner mask is sized and configured to define a positive pressure cavity for receiving the flow of breathing gas and convey the flow to the airway of the patient. The outer mask is sized and configured to define a negative pressure cavity that encompasses the inner mask and capture any gases escaping the inner mask.

### CROSS-REFERENCE TO RELATED APPLICATIONS

This patent application claims the priority benefit under 35 U.S.C. § 119(e) of U.S. Provisional Application No. 63/015,944, filed on Apr. 27, 2020, the contents of which are herein incorporated by reference.

### BACKGROUND OF THE INVENTION1

### 1 Field of the Invention

The present invention relates to non-invasive ventilation and pressure support systems in which a patient interface is used to deliver a flow of breathing gas to a patient and, more particularly, to a patient interface that minimizes gas passing from the mask to the ambient environment. The present invention also relates to a system adapted to provide a regimen of respiratory therapy to a patient that includes such patient interface.

### 2. Description of the Related Art

There are numerous situations where it is necessary or desirable to deliver a flow of breathing gas non-invasively to the airway of a patient, i.e., without intubating the patient or surgically inserting a tracheal tube the esophagus of the patient. For example, it is known to ventilate a patient using a technique known as non-invasive ventilation. It is also known to deliver positive airway pressure (PAP) therapy to treat certain medical disorders, such as obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Recently, PAP machines have also been utilized in treating patients suffering from certain diseases that adversely affect the patient's lungs such as Coronavirus (COVID-19). Gases expelled from such patients (e.g., via exhaling, coughing, sneezing) may be contaminated with the virus and thus can lead to infections to caregivers and others near the patient.

Non-invasive ventilation and pressure support therapies involve a gas flow generator to produce a flow of breathing gas, and the placement of a patient interface including a mask component on the face of a patient. The gas flow generator produces positive air pressure by taking air in from the surrounding environment and using a fan or other suitable arrangement to push the air out of the machine, through a delivery conduit, and into the patient interface to be delivered to the patient. Gases expelled from the patient are typically vented to the atmosphere via exhaust ports provided in the patient interface or on one or more components (e.g., an elbow conduit adjacent the patient interface) in the flow path between the gas flow generator and the patient interface. When used in treating a patient with a contagious or communicable disease, such arrangements would thus not address the infectious gases expelled from a patient, and instead may actually result in greater dispersion of such gases into the surrounding environment.

### SUMMARY OF THE INVENTION

As one aspect of the present invention a patient interface for use in delivering a flow of a breathing gas to an airway of a patient that includes an inner mask having an inner cushion with an inward curving inner sealing portion that is structured to sealingly engage the face of the patient about the mouth and nares of the patient. An outer mask is coupled to the inner mask. The outer mask has an outer cushion with an outward curving outer sealing portion that is structured to sealingly engage the face of the patient completely around, and outward from, the inner sealing portion of the inner mask. The inner mask is sized and configured to define a positive pressure cavity that is structured to receive the flow of breathing gas and convey the flow of breathing gas to the airway of the patient. The outer mask is sized and configured to define a negative pressure cavity that encompasses the inner mask. The negative pressure cavity is structured to be placed under negative pressure by a vacuum source fluidly connected to the negative pressure cavity.

As another aspect of the present invention, a patient interface for use in delivering a flow of a breathing gas to an airway of a patient includes a coupling conduit structured to receive the flow of breathing gas from a delivery conduit and an inner mask comprising an inner faceplate having an inlet port defined therethrough. The inlet port has a first portion of the coupling conduit received therein such that the inner faceplate, and thus the inner mask is coupled to the coupling conduit, and an inner cushion coupled to, and extending rearward generally from a periphery of the inner faceplate. The inner cushion has an inner sealing portion that is structured to sealingly engage the face of the patient about the mouth and nares of the patient. An outer mask comprising an outer faceplate has a primary port defined therethrough. The primary port has a second portion of the coupling conduit, which is further inward on the coupling portion than the first portion, received therein such that the outer faceplate, and thus the outer mask is coupled to the coupling conduit.

A vacuum port is defined through the outer faceplate, and an outer cushion coupled to, and extending rearward generally from, a periphery of the outer faceplate. The outer cushion has an outer sealing portion that is structured to sealingly engage the face of the patient completely around, and outward from, the inner sealing portion of the inner mask. The inner faceplate and the inner cushion define a positive pressure cavity that is structured to receive and convey the flow of breathing gas from the coupling conduit to the airway of the patient. The outer faceplate and the outer cushion define a negative pressure cavity encompassing the inner mask, wherein the vacuum port is structured to be coupled to a vacuum source that is structured to create a negative pressure in the negative pressure cavity, and wherein the negative pressure cavity is sized and configured to receive gases expelled and/or leaked from the positive pressure cavity of the inner mask.

As yet a further aspect of the present invention a patient interface for use in delivering a flow of a breathing gas to an airway of a patient comprises: a coupling conduit structured to receive the flow of breathing gas from a delivery conduit; an inner mask comprising: an inner faceplate having: a front side, a rear side disposed opposite the front side, and an inlet port defined through the inner faceplate, the inlet port having a first portion of the coupling conduit received therein such that the inner faceplate, and thus the inner mask is coupled to the coupling conduit; and an inner cushion extending rearward generally from a periphery of the inner faceplate, the inner cushion having: an inward curving inner sealing portion that is structured to sealingly engage the face of the patient about the mouth and nares of the patient, and an inner wall portion that is coupled to the inner faceplate and extends between the inner faceplate and the inner sealing portion; and an outer mask comprising: an outer faceplate having: a front side, a rear side disposed opposite the front side, a primary port defined through the outer faceplate, the primary port having a second portion of the coupling conduit, further inward on the coupling portion than the first portion, received therein such that the outer faceplate, and thus the outer mask is coupled to the coupling conduit, and a vacuum port defined through the outer faceplate; and an outer cushion extending rearward generally from a periphery of the outer faceplate, the outer cushion having: an outward curving outer sealing portion that is structured to sealingly engage the face of the patient completely around, and outward from, the inner sealing portion of the inner mask, and an outer wall portion that is coupled to the outer faceplate and extends between the outer faceplate and the outer sealing portion.

The inner faceplate and the inner cushion define a positive pressure cavity that is structured to receive and convey the flow of breathing gas from the coupling conduit to the airway of the patient, the outer faceplate and the outer cushion define a negative pressure cavity encompassing the inner mask, the vacuum port is structured to be coupled to a vacuum source that is structured to create a negative pressure in the negative pressure cavity, and the negative pressure cavity is sized and configured to receive gases expelled and/or leaked from the positive pressure cavity of the inner mask.

The inner mask may be nested within the outer mask.

The inner sealing portion may define a first opening that is structured to sealingly engage around the mouth of the patient and a second opening that is structured to sealingly engage around both nares of the patient.

The inner faceplate may further include a number of exhaust ports formed therein that are each sized and configured to allow passage of gas outward from the positive pressure cavity through the inner faceplate.

The coupling conduit may comprises an elbow conduit.

The inner faceplate and the outer faceplate may be formed from one or more polycarbonate materials.

The inner cushion and the outer cushion may be formed from silicone.

The outer faceplate may include a number of headgear engagement structures formed therein, each headgear engagement structure being sized and configured to cooperatively engage a strap of a headgear for securing the patient interface to the head of the patient.

Each headgear engagement structure may be formed with a corresponding window defined through the outer faceplate, and each window may be structured to allow for the passage of ambient air though the outer faceplate and into the negative pressure cavity.

The outer faceplate may include a number of bulged out regions wherein a spacing between the outer faceplate and the inner faceplate is greater than regions adjacent thereto.

One or both of the inner mask and or the outer mask may include one or more alignment features for aligning the inner mask and the outer mask with respect to each other.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a system (shown partially schematically) for providing a regiment of respiratory therapy to a patient according to one example embodiment of the present invention shown with an example patient interface thereof (according to one exemplary embodiment of the present invention) positioned on the face of a patient;

FIG. 2 is a top perspective view of the patient interface of FIG. 1 shown positioned on the face of a patient and with a coupling conduit connected thereto;

FIG. 3 is a bottom perspective view of the patient interface of FIG. 1 shown positioned on the face of a patient and with a coupling conduit connected thereto;

FIG. 4 is a side elevation view of the patient interface of FIG. 1 shown positioned on the face of a patient and with a coupling conduit connected thereto;

FIG. 5 is a rear elevation view of the patient interface of FIG. 1 show with a coupling conduit connected thereto;

FIG. 6 is a perspective view of an inner mask of the patient interface of FIGS. 1-5 shown positioned on the face of a patient;

FIG. 7 is a front elevation view of the inner mask of FIG. 6;

FIG. 8 is a top view of the inner mask of FIG. 6;

FIG. 9 is a front perspective view of the inner mask of FIG. 6;

FIG. 10 is a rear perspective view of the inner mask of FIG. 6;

FIG. 11 is a sectional view of the patient interface and coupling conduit taken along line 11-11 of FIG. 1;

FIG. 12 is a rear elevation view of an outer mask of the patient interface of FIGS. 1-5 shown with a coupling conduit connected thereto;

FIG. 13 is a rear perspective view of the outer mask and coupling conduit of FIG. 12;

FIG. 14 is a system (shown partially schematically) for providing a regiment of respiratory therapy to a patient according to one example embodiment of the present invention shown with a patient interface including the inner mask of FIGS. 6-10 positioned on the face of a patient;

FIG. 15 is a perspective view of the patient interface of FIG. 14 shown positioned on the face of a patient and with a coupling conduit connected thereto;

FIG. 16 is a side elevation view of the patient interface of FIG. 15 shown positioned on the face of a patient and with a coupling conduit connected thereto; and

FIG. 17 is a sectional view of the patient interface and coupling conduit of FIG. 14 taken along line 17-17 of FIG. 14.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are coupled directly in contact with each other (i.e., touching). As used herein, “fixedly coupled” or “fixed” means that two components are coupled so as to move as one while maintaining a constant orientation relative to each other.

As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, left, right, upper, lower, front, back, on top of, and derivatives thereof, relate to the orientation of the elements shown in the drawings and are not limiting upon the claims unless expressly recited therein.

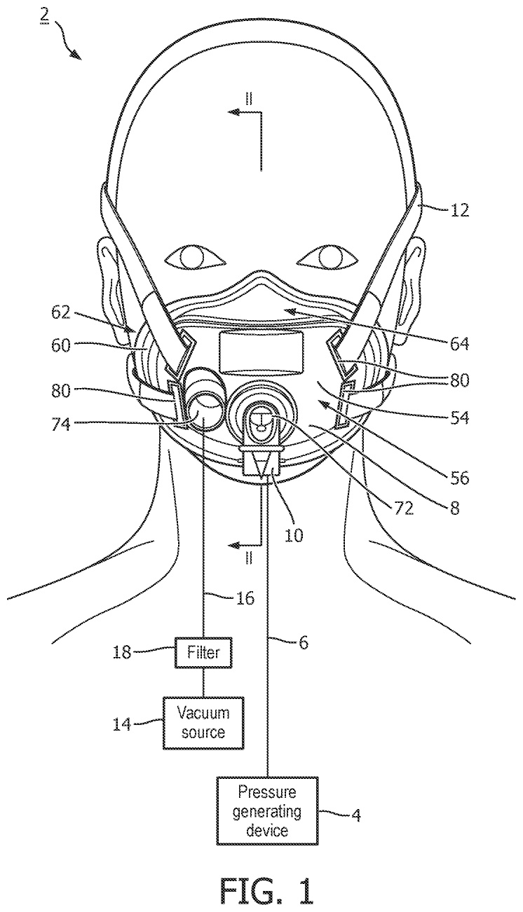

A system 2 adapted to provide a regimen of respiratory therapy to a patient according to one example embodiment of the invention is generally shown in FIG. 1. System 2 includes a pressure generating device 4 (shown schematically), a delivery conduit 6 (shown schematically), a patient interface 8 (shown disposed on the face of a patient, not numbered) having a fluid coupling conduit 10, and a headgear 12 (only portions of straps thereof are shown). Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is coupled between pressure generating device 4 and patient interface 8 and is structured to communicate the flow of breathing gas from pressure generating device 4 to patient interface 8 through fluid coupling conduit 10. Delivery conduit 6 and patient interface 8 are often collectively referred to as a patient circuit. In the exemplary embodiment illustrated in FIG. 1, fluid coupling conduit 10 is an elbow connector, however, it is to be appreciated that other suitable couplings may be employed without varying from the scope of the present invention. It is also to be appreciated that headgear 12 is provided solely for exemplary purposes and that any suitable headgear arrangement may be employed without varying from the scope of the present invention.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a patient or for elevating a pressure of gas at an airway of the patient, including the pressure support systems summarized above and non-invasive ventilation systems.

System 2 further includes a vacuum source 14 and vacuum conduit 16. Vacuum source is 14 is structured to create a vacuum in a selected portion of patient interface 8 (discussed in greater detail below) for removing gases expelled by a patient from patient interface 8. Vacuum source 14 may be any suitable source of vacuum such as, for example, without limitation, a vacuum port on a large scale vacuum system (e.g., a hospital vacuum system), an inlet port on a pressure generating device (e.g., another device the same as, or similar to pressure generating device 4), or any other suitable source of a generally high flow, low negative pressure vacuum.

As discussed in greater detail below, vacuum conduit 16 is coupled between vacuum source 14 and patient interface 8 and is structured to communicate a flow of gas expelled by the patient from patient interface 8 to vacuum source 14. Vacuum conduit 16 may include a replaceable filter 18 provided therein or at an end thereof for selectively filtering gas expelled from the patient before reaching vacuum source 14. For example, filter 18 may be formed from a suitable material or materials to prevent the transmission of contaminated particles expelled from a patient from passing to vacuum source 14 and further to the surrounding environment.

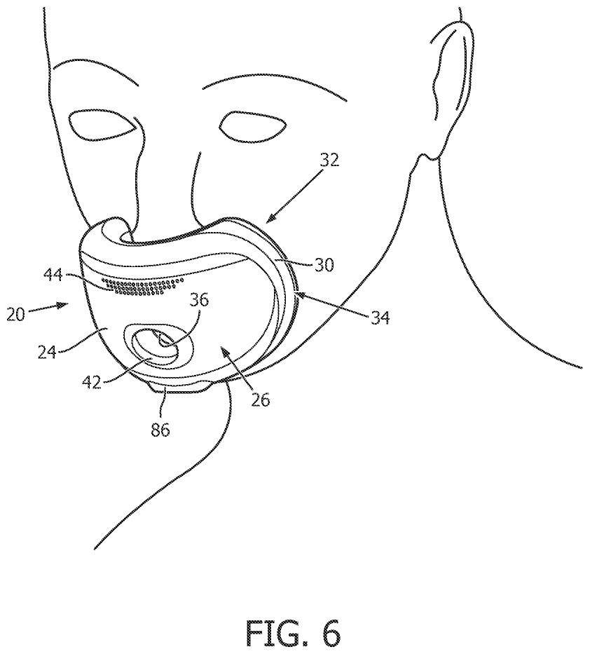

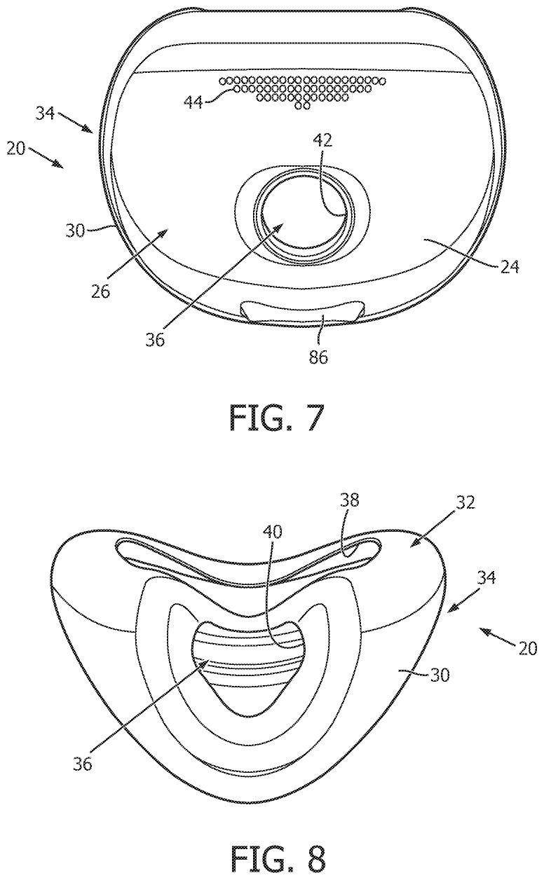

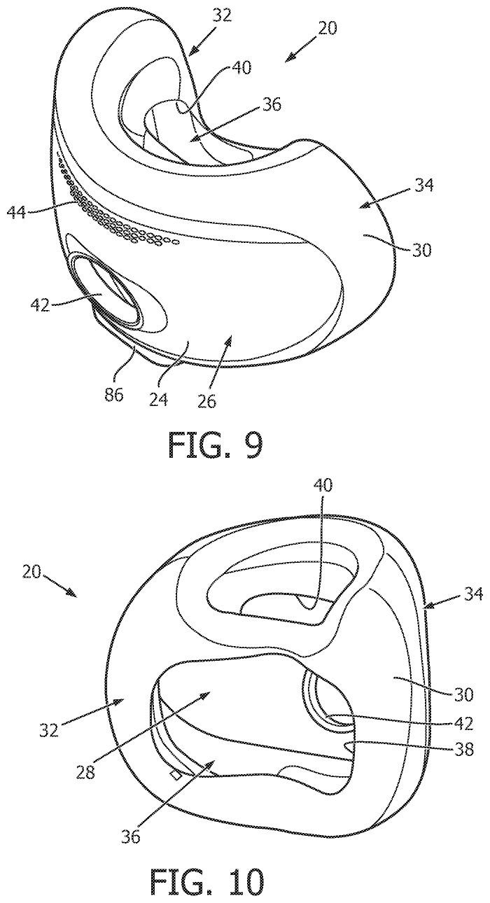

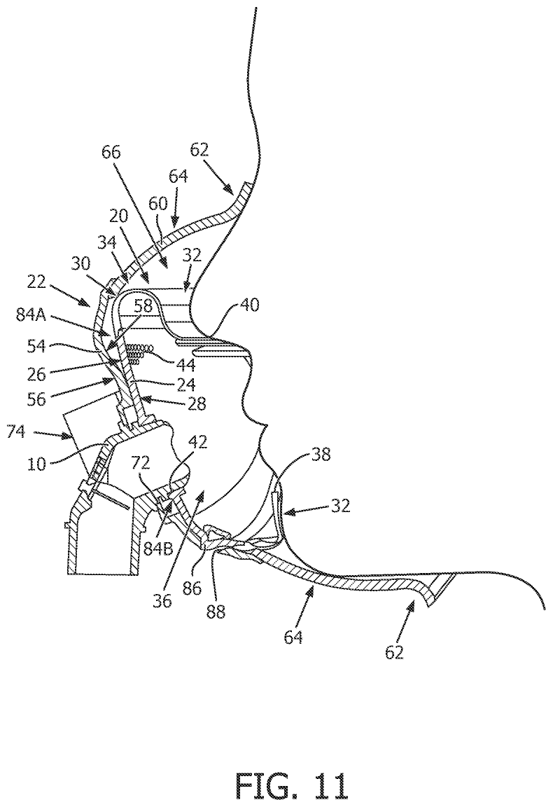

As shown in FIGS. 5 and 11, patient interface 8 includes an inner mask 20 that is coupled to, and generally nested within, an outer mask 22 or skirt. Continuing to refer to FIGS. 5 and 11, as well as to FIGS. 6-10, inner mask 20 includes a generally rigid inner faceplate 24 having a front side 26 and a patient-facing rear side 28 disposed opposite front side 26, and an inner cushion 30 extending rearward generally from the periphery of inner faceplate 24 for sealingly engaging the face of the patient. Inner faceplate 24 may be formed from a polycarbonate or other suitable rigid or semi-rigid material. Inner cushion 30 may be formed from silicone or any other suitable flexible material. Inner cushion 30 includes an inward curving inner sealing portion 32 that is structured to sealingly engage the face of the patient and an inner wall portion 34 that is coupled to inner faceplate 24 and extends between inner faceplate 24 and inner sealing portion 32. Together, inner faceplate 24 and inner cushion 30 define a positive pressure cavity 36 for receiving and conveying the flow of breathing gas produced by pressure generating device 4 to the airway of a patient.

In one example embodiment, such as illustrated herein, inner sealing portion 32 defines a first opening 38 that is structured to sealingly engage around the mouth of a patient and a second opening 40 that is structured to sealingly engage around both nares of the patient. It is to be appreciated, however, that inner sealing portions of other arrangements, e.g., one opening surrounding both the mouth and nares, three individual openings surrounding the mouth and each nare, an opening surrounding the mouth and a pair of nasal pillows, may be employed without varying from the scope of the present invention.

Inner faceplate 24 includes an inlet port 42 that is sized and configured to receive, and be coupled to, coupling conduit 10 for receiving the flow of breathing gas communicated from pressure generating device 4 into positive pressure cavity 36. Inner faceplate 24 further includes a number of exhaust passages or ports 44 formed therein that are each sized and configured to allow passage of gas outward from positive pressure cavity 36 through inner faceplate 24.

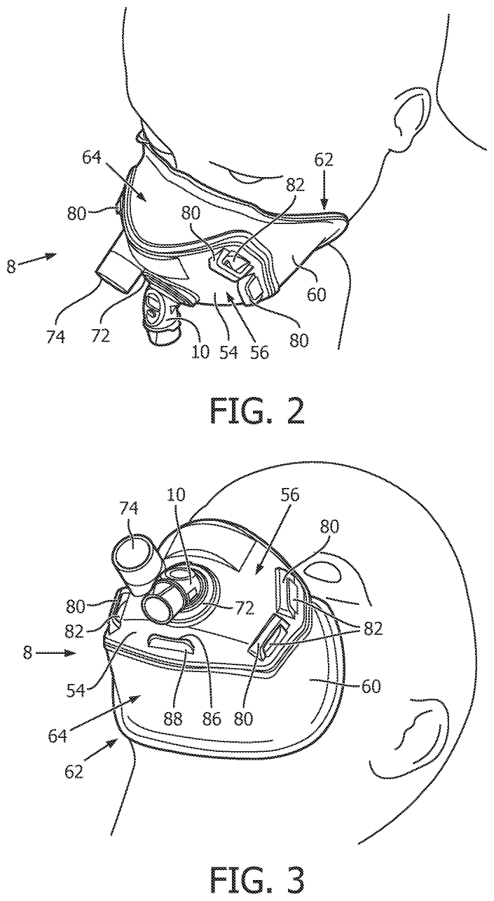

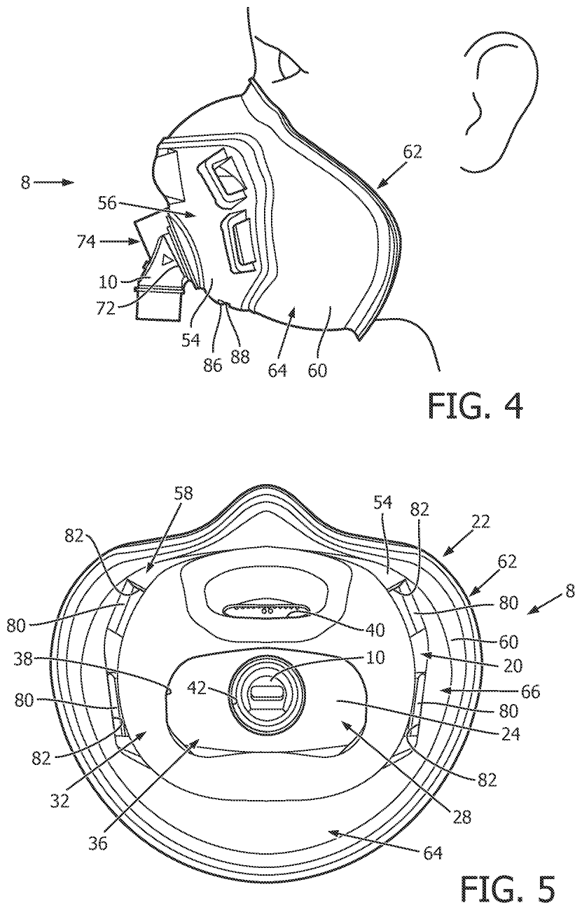

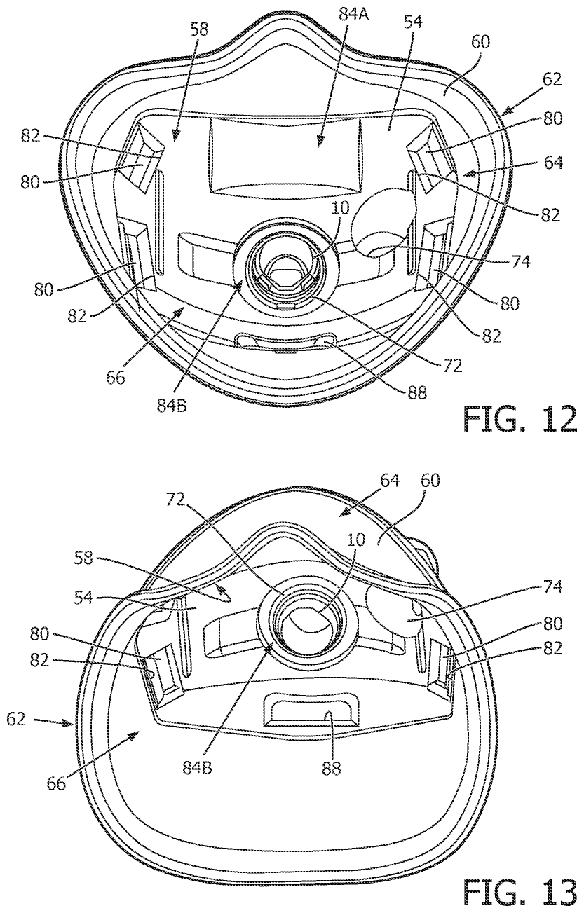

Referring now to FIGS. 1-5 and 11-13, outer mask 22 includes a generally rigid outer faceplate 54 having a front side 56 and a patient-facing rear side 58 disposed opposite front side 56, and an outer cushion 60 extending rearward generally from the periphery of outer faceplate 54 for sealingly engaging the face of the patient. Outer faceplate 54 may be formed from a polycarbonate or other suitable rigid or semi-rigid material. Outer cushion 60 may be formed from silicone or any other suitable flexible material. Outer cushion 60 includes an outward curving outer sealing portion 62 that is sized and configured to sealingly engage the face of the patient completely around and outward from inner sealing portion 32 of inner mask 20, and an outer wall portion 64 that is coupled to outer faceplate 54 and extends between outer faceplate 54 and outer sealing portion 62. Together, outer faceplate 54 and outer cushion 60 define a negative pressure cavity 66 encompassing inner mask 20 for receiving/capturing gases expelled and/or leaked from inner mask 20, and more particularly from positive pressure cavity 36 of inner mask 20. Such gases received/captured by negative pressure cavity 66 include gases exhausted through any of exhaust ports 44, leakage gases from between inner sealing portion 32 and the skin of the patient, and leakage gases from the connection between coupling conduit 10 and inner faceplate 24. Hence, it is to be appreciated that outer mask 22 is sized and configured to capture any potentially contaminated gases expelled from the nose or mouth of a patient receiving treatment from patient interface 8.

Continuing to refer to FIGS. 1-5 and 11-13, outer faceplate 54 includes a primary port 72 that is sized and configured to receive therethrough, and be coupled to, coupling conduit 10 at a location further inward on coupling conduit 10 from the location of the connection between coupling conduit 10 and inner faceplate 24 of inner mask 20 previously discussed. Hence, primary port 72 is provided to allow for passage of coupling conduit 10 through outer faceplate 54 and onto inner mask 20, as well as to provide for coupling outer mask 22 to inner mask 20. Outer faceplate 54 further includes a vacuum port 74 defined therethrough that is sized and configured to be coupled to vacuum conduit 16 (FIG. 1) such that a negative pressure is created within negative pressure cavity 66 between inner mask 20 and outer mask 22 by vacuum source 14 (FIG. 1). Through such arrangement, all gases expelled from inner mask 20, whether intentionally (i.e., exhaust) or unintentionally (i.e., leakage) are captured and removed from patient interface 8 via vacuum conduit 16.

In addition to the features previously described, outer mask 22 may further include one or more additional features in accordance with various example embodiments of the present invention. For example, outer faceplate 54 may include a number of headgear engagement structures 80 (four are shown in the example embodiment illustrated) for cooperatively engaging straps of headgear 12 in securing patient interface 8 to the head of a patient. Such structures may generally be of any suitable size and shape without varying from the scope of the present invention. In the example embodiment illustrated herein, each headgear engagement structure 80 is formed with a corresponding window 82 defined through outer faceplate 54. Such windows 82 provide for integral molding of each headgear engagement structure 80 with outer faceplate 54 as well as generally predetermined leakage points into negative pressure cavity 66, thus preventing undesirable over buildup of negative pressure within negative pressure cavity 66 due to a lack of gases passing into negative pressure cavity 66 (e.g., low amount of exhaust gases from patient, little to no leakage into negative pressure cavity 66).

This configuration allows for the outer mask to be used with a conventional inner mask. When there is no need to collect the exhaust gas, the patient would use only the inner mask as is conventional in non-invasive ventilation, positive pressure therapy, OSA treatment, or any other situation where a flow of gas is being delivered to the patient via a mask or circuit with an exhaust port to atmosphere. When there is need to collect the exhaust gas, e.g., when treating a patient with a communicable disease, such as COVID-19), the outer mask can be selectively attached to the inner mask and a negative pressure (vacuum) applied to the chamber in between. Thus, the present invention provides a highly adaptable system for treating infectious and non-infectious patients, and mask uses of existing positive pressure masks so that in an emergency pandemic, the existing supply of inner masks can be used and there need only be the need to provide the outer mask.

As another example, outer faceplate 54 may include one or more bulged out regions where the spacing between outer faceplate 54 and inner faceplate has been increased so as to promote flow into negative pressure cavity 66. The example shown in FIGS. 11-13 includes generally two of such regions 84A and 84B, a first region 84A is defined generally at or about exhaust ports 44 of inner faceplate 24, and a second region 84B is defined at or about inlet port 42 of inner faceplate 24 and extends outward therefrom. As yet a further example, one or both of inner mask 20 and outer mask 22 may include one or more alignment features for aligning inner mask 20 and outer mask 22 with respect to each other. In the example illustrated, inner faceplate 24 of inner mask 20 includes a protrusion 86 extending generally from a lower portion thereof, while outer faceplate 54 of outer mask 22 includes a cooperatively shaped opening 88 formed in a lower portion thereof. In addition to providing for aligning/clocking of inner mask 20 and outer mask 22, opening 88 also provides for a further predetermined leakage pathway into negative pressure cavity 66.

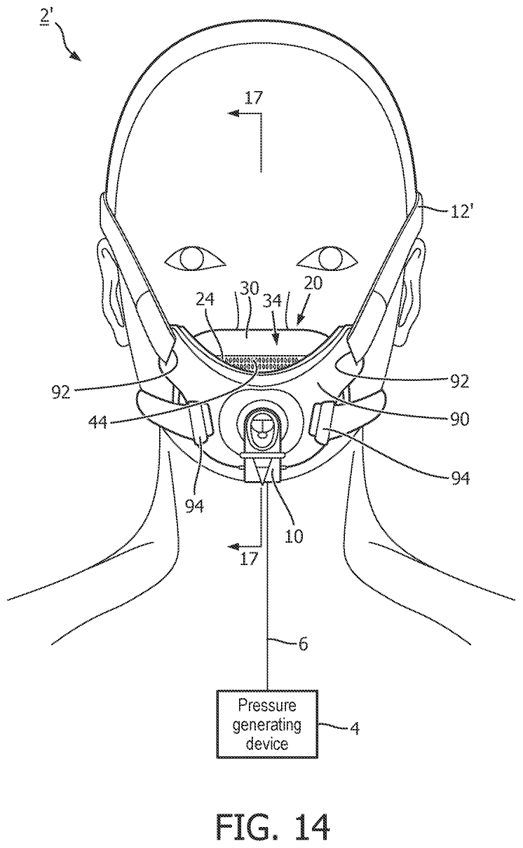

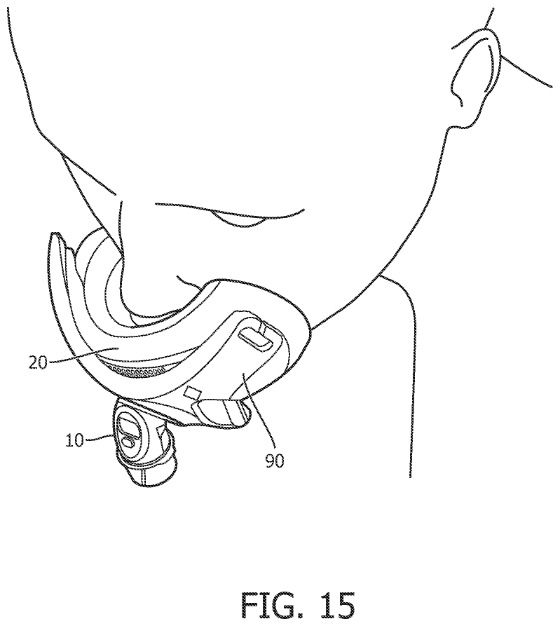

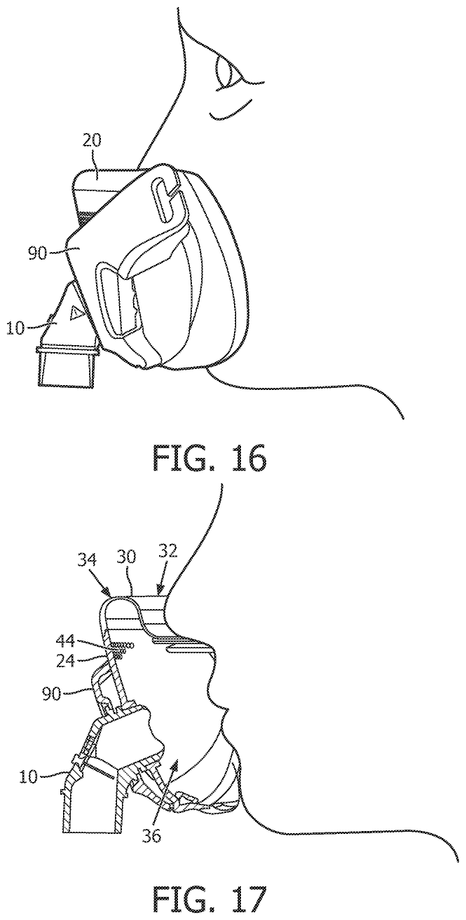

As shown in the one example embodiment of FIGS. 14-17, inner mask 20 may also be utilized without outer mask 22 in a system 2′ similar to system 2 previously discussed in regard to FIG. 1. In such example embodiment, a frame 90 has been employed in place of outer mask 22. Frame 90 includes a number of suitable structures 92 and 94 (two of each are shown in the example) for coupling with a suitable headgear 12′ in order to secure inner mask 20 to a patient. Hence, it is to be appreciated that the present invention contemplates arrangements utilizing an inner mask that may be used without an outer mask in treating a patient as well as an outer mask that may be used generally as a retrofit over a conventional mask in providing an improved patient interface arrangement that safely evacuates potentially contaminated gases expelled from a patient safely away from the patient and their surroundings and those thereby.

It should be noted that in the illustrated exemplary embodiment, outer mask 22 attached to inner mask 20 by the connection of coupling conduit 10 within inlet port 42 of inner faceplate 24. Outer mask 22 is carried on or already attached to conduit coupling 10 so that then conduit 10 is snapped or otherwise engaged to the inner mark, the completed assembly including the inner and outer masks is formed. To switch to the conventional assembly, an coupling conduit that does not include the outer mask can be provided.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A patient interface for use in delivering a flow of a breathing gas to an airway of a patient, the patient interface comprising:
an inner mask having an inner cushion with an inward curving inner sealing portion that is structured to sealingly engage the face of the patient about the mouth and nares of the patient, and
an outer mask coupled to the inner mask, the outer mask having an outer cushion with an outward curving outer sealing portion that is structured to sealingly engage the face of the patient completely around, and outward from, the inner sealing portion of the inner mask,
wherein the inner mask is sized and configured to define a positive pressure cavity that is structured to receive the flow of breathing gas and convey the flow of breathing gas to the airway of the patient, wherein the outer mask is sized and configured to define a negative pressure cavity that encompasses the inner mask, and wherein the negative pressure cavity is structured to be placed under negative pressure by a vacuum source fluidly connected to the negative pressure cavity.

2. A patient interface for use in delivering a flow of a breathing gas to an airway of a patient, the patient interface comprising:
(a) a coupling conduit structured to receive the flow of breathing gas from a delivery conduit;
(b) an inner mask comprising:
an inner faceplate having an inlet port defined therethrough, the inlet port having a first portion of the coupling conduit received therein such that the inner faceplate, and thus the inner mask is coupled to the coupling conduit, and
an inner cushion coupled to, and extending rearward generally from, a periphery of the inner faceplate, the inner cushion having an inner sealing portion that is structured to sealingly engage the face of the patient about the mouth and nares of the patient; and
(c) an outer mask comprising:
an outer faceplate having a primary port defined therethrough, the primary port having a second portion of the coupling conduit, further inward on the coupling portion than the first portion, received therein such that the outer faceplate, and thus the outer mask is coupled to the coupling conduit, and a vacuum port defined through the outer faceplate, and
an outer cushion coupled to, and extending rearward generally from, a periphery of the outer faceplate, the outer cushion having an outer sealing portion that is structured to sealingly engage the face of the patient completely around, and outward from, the inner sealing portion of the inner mask,
wherein the inner faceplate and the inner cushion define a positive pressure cavity that is structured to receive and convey the flow of breathing gas from the coupling conduit to the airway of the patient, wherein the outer faceplate and the outer cushion define a negative pressure cavity encompassing the inner mask, wherein the vacuum port is structured to be coupled to a vacuum source that is structured to create a negative pressure in the negative pressure cavity, and wherein the negative pressure cavity is sized and configured to receive gases expelled and/or leaked from the positive pressure cavity of the inner mask.

3. A patient interface for use in delivering a flow of a breathing gas to an airway of a patient, the patient interface comprising:
(a) a coupling conduit structured to receive the flow of breathing gas from a delivery conduit;
(b) an inner mask comprising:
(1) an inner faceplate having:
a front side,
a rear side disposed opposite the front side, and
an inlet port defined through the inner faceplate, the inlet port having a first portion of the coupling conduit received therein such that the inner faceplate, and thus the inner mask is coupled to the coupling conduit; and
(2) an inner cushion extending rearward generally from a periphery of the inner faceplate, the inner cushion having:
an inward curving inner sealing portion that is structured to sealingly engage the face of the patient about the mouth and nares of the patient, and
an inner wall portion that is coupled to the inner faceplate and extends between the inner faceplate and the inner sealing portion; and
(c) an outer mask comprising:
(1) an outer faceplate having:
a front side,
a rear side disposed opposite the front side,
a primary port defined through the outer faceplate, the primary port having a second portion of the coupling conduit, further inward on the coupling portion than the first portion, received therein such that the outer faceplate, and thus the outer mask is coupled to the coupling conduit, and
a vacuum port defined through the outer faceplate; and
(2) an outer cushion extending rearward generally from a periphery of the outer faceplate, the outer cushion having:
an outward curving outer sealing portion that is structured to sealingly engage the face of the patient completely around, and outward from, the inner sealing portion of the inner mask, and
an outer wall portion that is coupled to the outer faceplate and extends between the outer faceplate and the outer sealing portion,
wherein the inner faceplate and the inner cushion define a positive pressure cavity that is structured to receive and convey the flow of breathing gas from the coupling conduit to the airway of the patient,
wherein the outer faceplate and the outer cushion define a negative pressure cavity encompassing the inner mask, wherein the vacuum port is structured to be coupled to a vacuum source that is structured to create a negative pressure in the negative pressure cavity, and wherein the negative pressure cavity is sized and configured to receive gases expelled and/or leaked from the positive pressure cavity of the inner mask.

4. The patient interface of claim 3, wherein the inner mask is nested within the outer mask.

5. The patient interface of claim 3, wherein the inner sealing portion defines a first opening that is structured to sealingly engage around the mouth of the patient and a second opening that is structured to sealingly engage around both nares of the patient.

6. The patient interface of claim 3, wherein the inner faceplate further includes a number of exhaust ports formed therein that are each sized and configured to allow passage of gas outward from the positive pressure cavity through the inner faceplate.

7. The patient interface of claim 3, wherein the coupling conduit comprises an elbow conduit.

8. The patient interface of claim 3, wherein the inner faceplate and the outer faceplate are formed from one or more polycarbonate materials.

9. The patient interface of claim 3, wherein the inner cushion and the outer cushion are formed from silicone.

10. The patient interface of claim 3, wherein the outer faceplate includes a number of headgear engagement structures formed therein, each headgear engagement structure being sized and configured to cooperatively engage a strap of a headgear for securing the patient interface to the head of the patient.

11. The patient interface of claim 10, wherein each headgear engagement structure is formed with a corresponding window defined through the outer faceplate, and wherein each window is structured to allow for the passage of ambient air though the outer faceplate and into the negative pressure cavity.

12. The patient interface of claim 3, wherein the outer faceplate includes a number of bulged out regions wherein a spacing between the outer faceplate and the inner faceplate is greater than regions adjacent thereto.

13. The patient interface of claim 3, wherein one or both of the inner mask and or the outer mask include one or more alignment features for aligning the inner mask and the outer mask with respect to each other.
